(19) United States
(12) Patent Application Publication
(10) Pub. No.: US 2013/0143230 A1
(43) Pub. Date: Jun. 6, 2013

(54) MICROFLUIDIC-BASED CELL-CULTURING PLATFORM AND METHOD

(71) Applicant: The Trustees of the Stevens Institute of Technolog, Hoboken, NJ (US)

(72) Inventors: Peter Tolias, Westfield, NJ (US); Woo Young Lee, Ridgewood, NJ (US); Arthur B. Ritter, Morristown, NJ (US); Xiaojun Yu, Fishers, IN (US); Hongjun Wang, Millburn, NJ (US); Henry Du, Short Hills, NJ (US); Chang-Hwan Choi, Palisades Park, NJ (US); Wenting Zhang, Jersey City, NJ (US); Yexin Gu, North Bergen, NJ (US)

(73) Assignee: The Trustees of the Stevens Institute of Technology, Hoboken, NJ (US)

(21) Appl. No.: 13/690,831

(22) Filed: Nov. 30, 2012

Related U.S. Application Data
(60) Provisional application No. 61/566,261, filed on Dec. 2, 2011.
(60) Provisional application No. 61/568,811, filed on Dec. 9, 2011.

(51) Int. Cl. C12Q 1/02

(52) U.S. Cl. CPC C12Q 1/025
USPC 435/7.1; 435/289.1; 435/297.2; 435/288.7; 435/287.2; 435/34

(57) ABSTRACT

A microfluidic-based platform with cultured three-dimensional tissues simulates major human physiological systems for rapid evaluation of individual drugs prior to clinical testing or for personalized medical applications. The platform integrates the circulatory and lymphatic systems in a physiologically correct manner. The physiological systems may be simulated in the platform by microfluidic tissue culture devices which accommodate various tissues and provide integrated microvascular and lymphatic systems. Biomimetic nanofiber meshes or microfiber structures may be used to provide the cells with a physiologically relevant substrate. Each device may have an on-board detection system utilizing optical fiber bundles for microarray multiplexing of biomarkers, label-free SERS measurement of drugs, and microendoscopic confocal imaging of cells and tissues.

### CROSS-REFERENCE TO RELATED APPLICATIONS

The present application claims the benefit of U.S. Provisional Patent Application No. 61/566,261, filed on Dec. 2, 2011, which is incorporated herein in its entirety, and the benefit of U.S. Provisional Patent Application No. 61/568,811, filed on Dec. 9, 2011, which is also incorporated herein in its entirety.

### FIELD OF THE INVENTION

The present invention relates to microfluidic cell-culturing platforms, and more particularly to microfluidic cell-culturing platforms for: (i) culturing cells in three-dimensions; (ii) simulation of human physiological systems; (iii) drug development and evaluation, and (iv) drug therapy selection.

### BACKGROUND OF THE INVENTION

Assessing the efficacy of drugs typically involves a series of investigations in various investigation platforms. For example, in vitro testing may be used in the early stages of an investigation, wherein the platform is a two-dimensional (2D) layer of cultured cells. Animal tests may follow, which may then lead to clinical trials in humans. It has often been observed that drug therapies which show promise during the in vitro test platform disappointing results in the animal testing or clinical trial phases. Conventional 2D culturing techniques have characteristics which may contribute to this disparity between the effectiveness observed in vitro and the inadequate performance encountered in the in vivo phases. For example, 2D cultures often test the effects of the drugs on a single cell layer or tissue type and do not account for the perfusion of drugs through a three-dimensional (3D) tissue. Also. unlike the case when cells grow within a 3D tissue environment, cells grown in 2D culture undergo changes in cell shape as they become elongated on the surface of the culture chamber. This leads to changes in the organization of the cytoskeleton, global gene expression, and the molecular physiology of the cell, altering its accessibility to drugs. Further, 2D cultures cannot simulate the interactions of physiological systems in the animal or human body.

### SUMMARY OF THE INVENTION

In one aspect, the present invention provides a scalable investigation platform which simulates functions of major human physiological systems for the rapid evaluation of drug safety, efficacy and pharmacokinetics prior to clinical trials. In an embodiment of the present invention, the platform comprises one or more 3D tissue culture chambers with flows and volumes designed to represent the fraction of cardiac output and residence times present under normal homeostatic physiological conditions. In another embodiment of the present invention, the chambers are fluidly interconnected to evaluate interactions between different physiological systems. In an embodiment, the addition of the lymphatic and portal systems to the tissue culture chambers mimics cardiovascular models of circulation, allowing for physiologically accurate assessment of drug and vaccine distribution, utilization and elimination in the animal or human body.

In another aspect, the present invention provides microfluidic devices for the culturing and testing of three-dimensional tissues and the simulation of physiological systems. In an embodiment of the present invention, the devices are scalable elements of the platform, and accommodate various tissues with integrated microvascular and lymphatic systems. In embodiments of the present invention, biomimetic nanofiber meshes are used within the devices to provide the cells with physiologically relevant barriers, substrates, and scaffolds, and in particular, mimic the basement membrane structure of epithelial and endothelial layers. In embodiments of the present invention, microfibers are used to mimic the reticular connective tissue in lymph nodes to anchor phagocytes and lymphocytes.

In yet another aspect, the present invention, employs an on-board data acquisition system to monitor the progress of the investigation. In an embodiment, the on-board data acquisition system may be arranged in two areas: (1) the integration of fiber optic bundles containing encoded microspheres coated with specific capture molecules (e.g., antibodies) or surface-enhanced Raman scattering (SERS)-active gold (Au) nanoparticles, for multiplex measurements of drugs, biomarkers, and other critical indicators of interest, and (2) the integration of micro-lens-tipped fiber optic bundles that enable real-time confocal imaging of cells and tissues.

### BRIEF DESCRIPTION OF THE DRAWINGS

For a more complete understanding of the present invention, reference is made to the following detailed description of exemplary embodiments thereof, considered in conjunction with the accompanying drawings, in which:

FIG. 1 is a schematic diagram of a conceptual framework for implementing embodiments of the present invention;

FIG. 2 is a photograph of an exemplary investigation platform according to an embodiment of the present invention;

FIG. 3 is a schematic drawing of an exemplary three-dimensional (3D) tissue culturing device according to an embodiment of the present invention;

FIG. 4 is a top-down view of a portion of the platform of FIG. 2;

FIG. 5 is a schematic drawing of a tissue culturing device of FIG. 3 showing a cultured 3D tissue;

FIG. 6 is a schematic drawing of a second exemplary 3D tissue culturing device according to an embodiment of the present invention in a partially-assembled state;

FIG. 7 is a schematic drawing of the exemplary 3D tissue culturing device of FIG. 6 in a fully-assembled state;

FIG. 8 is a schematic drawing of a third exemplary 3D tissue culturing device according to an embodiment of the present invention in a partially-assembled state;

FIG. 9 is a schematic drawing of the exemplary 3D tissue culturing device of FIG. 8 in a fully-assembled state;

FIG. 10 is a schematic drawing of a bottom-up view of a portion of the exemplary tissue culturing device of FIG. 8;

FIG. 11 is a schematic drawing of a vertical cross-section of a cell-seeded portion of the exemplary 3D tissue culturing device of FIGS. 8 and 9;

FIG. 12 is a schematic drawing of a vertical cross-section of a second cell-seeded portion of the exemplary 3D tissue culturing device of FIGS. 8 and 9;

FIG. 13 is a schematic drawing of a vertical cross-section of a third cell-seeded portion of the exemplary 3D tissue culturing device of FIGS. 8 and 9;

FIG. 14 is a schematic drawing of a fourth exemplary 3D tissue culturing device according to an embodiment of the present invention in a partially-assembled state;

FIG. 15 is a schematic drawing of the exemplary 3D tissue culturing device of FIG. 14 in a fully-assembled state;

FIG. 16 is schematic drawing of a bottom-up view of a portion of an exemplary 3D tissue culturing device similar to that of FIG. 8, to which optical fiber sensors have been added according to an embodiment of the present invention;

FIG. 17 is a schematic drawing of an enlarged view of the end of an optical fiber sensor of FIG. 16; and

FIG. 18 is a schematic drawing of an enlarged view of the end of a functionalized optical fiber of the optical fiber sensor of FIG. 17.

### DETAILED DESCRIPTION OF THE EXEMPLARY EMBODIMENTS

The present invention may be described, generally, as a microfluidic-based three-dimensional tissue-culturing platform that functionally represents major animal physiological systems (including human systems) for rapid evaluation of drug safety, efficacy and pharmacokinetics prior to clinical trials. In some embodiments, the platform can be used for generic assessment of individual drugs, or in personalized medicine applications to evaluate single drugs or drug cocktails where cells of all physiological systems are derived from a single patient. In some embodiments, the platform enables the introduction, growth and maintenance of different types of immortalized cell lines, as well as primary cells, as three-dimensional (“3D”) tissues and the introduction of drugs to be evaluated. Suitable immortalized cell lines, as well as primary cells can be obtained from commercial and academic cell banks, or from the patient's body. The platform may include an on-board detection system that reports on drug toxicity and efficacy by providing pharmacokinetic or pharmacodynamic data necessary for accurate modeling and simulation of animal or human physiological responses to the assessed drug(s). The use of one or more multiple tissue-culturing devices contributes to the sealability of the platform. The platform and devices are described in further detail hereinbelow. For the purposes of this disclosure and the appended claims, the general term “animal” includes human beings.

### Architectural Design of the Platform

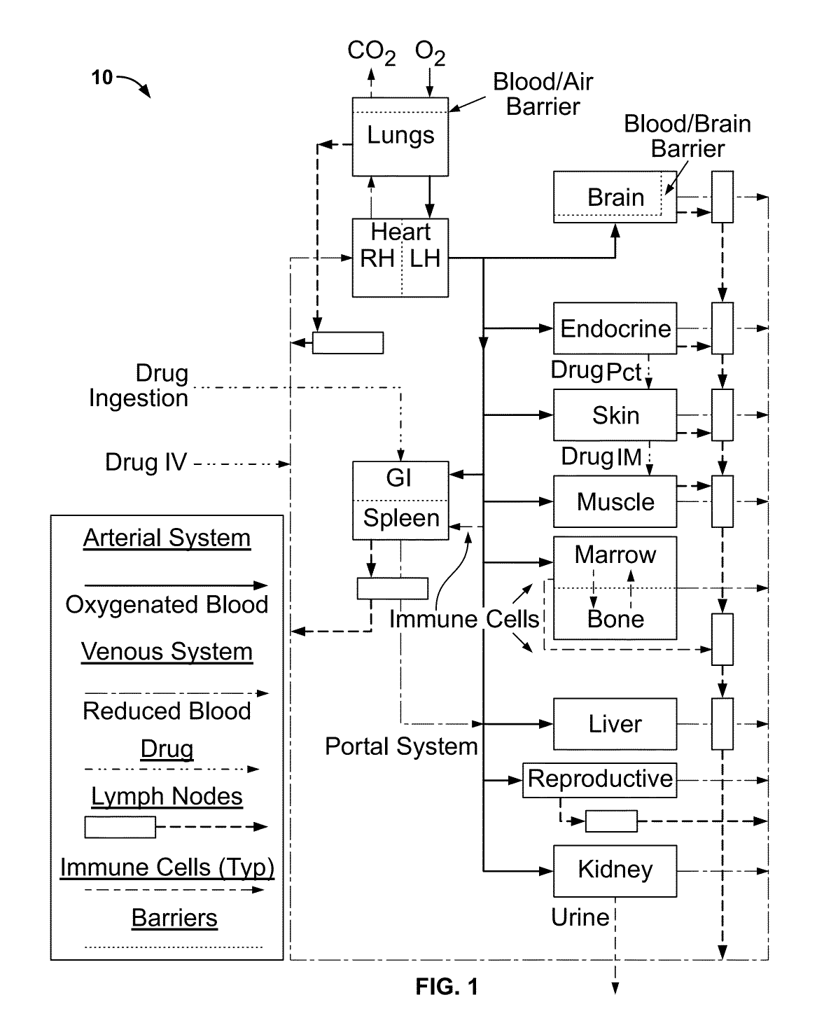

FIG. 1 is a schematic diagram of a conceptual framework 10 for implementing embodiments of the present invention, with reference to the major organs of the animal body, and their interactions and interconnectivity with respect to the flow of blood, volume of distribution, and the generation of lymph. The major routes by which drugs are introduced into the body are also represented. In FIG. 1, the major organs are identified by name and the systemic interaction and interconnectivity of these organs are illustrated by arrows. Routes for introduction of drugs are also identified by name and by arrows. Barriers associated with the various organs (e.g., the blood/brain barrier or the blood/air barrier) are indicated by dotted lines.

Exemplary physiological systems which can be evaluated in embodiments of the platform include: the lung; the heart (right heart [RH] and left heart [LH]); the brain; the endocrine system; the skin; muscle; bone and marrow; the liver; the reproductive system; the kidney; and the gastrointestinal (GI) system including the spleen. The framework of FIG. 1 also includes the lymphatic system, the flow or migration of immune cells derived from bone marrow, gas exchange in the lungs, and the excretion of urine. These systems and functions may also be simulated in three-dimensional cell-culturing platforms made and operated according to embodiments of the present invention.

The framework 10 also includes entry points where drugs can be administered in the animal body and the platform. These entry points are depicted by the dotted arrows 18, which simulate: drug ingestion into the GI system; drug penetration (Pct) by absorption through the skin; drug intramuscular (IM) injection; and drug intravenous (IV) injection.

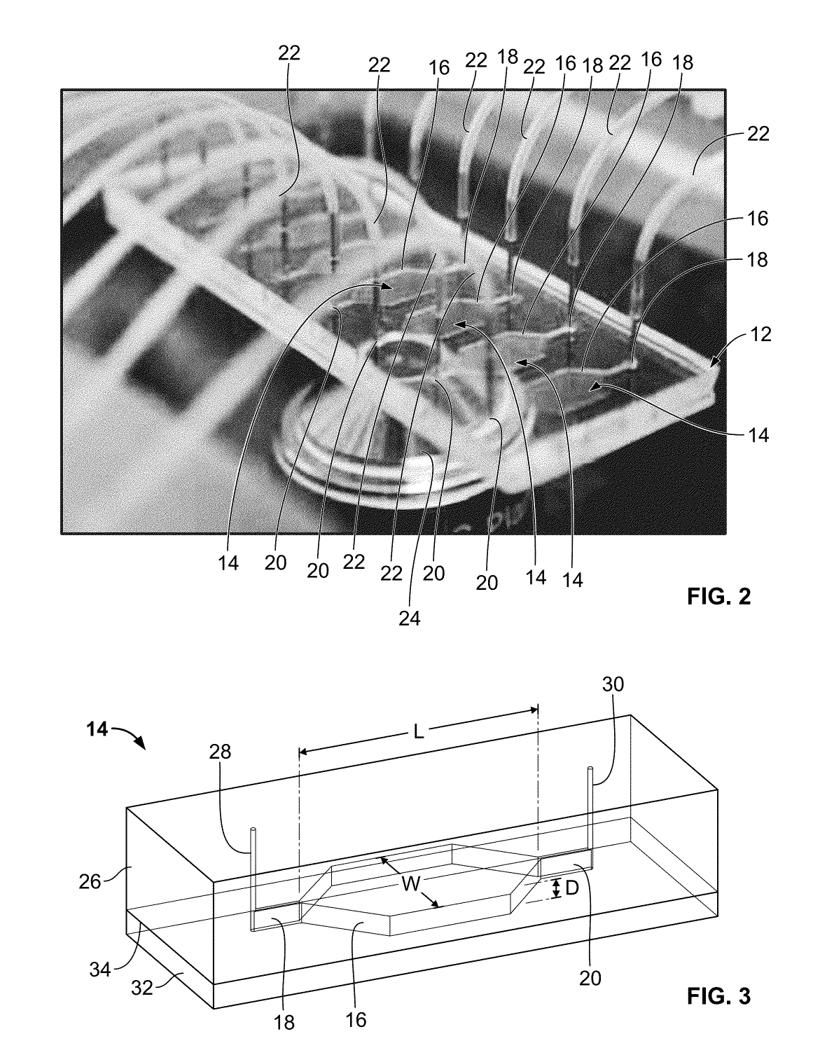

Referring to FIG. 2, in certain embodiments of the present invention, the platform 12 for implementing the framework 10 of FIG. 1 is a platform of interconnected 3D microfluidic tissue culture devices 14 having tissue culture chambers 16. FIG. 2 is a photograph of an embodiment of such a platform 12. An exemplary platform 12 arranged according to certain embodiments of the present invention comprises a plurality of such devices 14. Each device 14 includes at least one tissue culture chamber 16 wherein cells may be cultured to form three-dimensional tissues. Each chamber 16 has an inlet 18 and an outlet 20. Capillary tubing 22 are connected to the inlet 18 and outlet 20 to provide a flow of culture medium or other fluids (e.g., fluids represented in framework 10) through the chamber 16 (also refer to FIG. 3, discussed below). FIG. 2 also shows the objective of a microscope 24, which may be provided to obtain images (“imaging”) of the chambers 16. In other embodiments of the present invention, physiological systems are represented by devices (not shown) that are more complex and sophisticated than the devices of FIG. 2. Such devices are discussed elsewhere hereinbelow. Yet other embodiments of the present invention may use imaging devices other than that shown in FIG. 2, as is also discussed further herein below.

In embodiments of the present invention, liquids are pumped through the devices 14 at flow rates and volumes designed to represent the fraction of cardiac output (i.e., total medium flow rate) and residence time (i.e., volume/flow) present under normal homeostatic physiological conditions, including the integration of the cardiovascular and lymphatic systems in a physiologically correct manner. The desired flow rates may be provided by the use of microfluidic pumps (not shown).

FIG. 3 is schematic drawing of a device 14 such as that used in the platform 12 of FIG. 2. The device 14 comprises the tissue culture chamber 16 formed in an upper layer 26 of the device 14, and an inlet 18 and an outlet 20 of the chamber 16 which are also formed in the upper layer 26. The chamber 16 has the shape of an elongated hexagonal prism, although chambers having other shapes may be used in other embodiments of the present invention. Bores 28, 30 in the upper layer 26 provide fluid communication between the inlet 18 and outlet 20 and the rest of the platform 12. In use, the capillary tubing of FIG. 2 are fluidly connected to the inlet 18 and outlet 20 of the chamber 16 via the bores 28, 30. The chamber 16 is completed by a lower layer 32 sealed to the upper layer 26. It is to be understood that the terms “lower” and “upper” as used in the present disclosure refer to the relative orientation of the layers 26, 32, and their counterparts in other embodiments, as shown in the drawing, and do not necessarily refer to the relative orientation of the elements of the device 14 and its counterpart in other embodiments, when used according to embodiments of the present invention.

The device 14 may be made by adapting methods presently used in the art for making biological and fluidic devices. For example, continuing to refer to FIG. 3, the device 14 may be made by making a positive relief of the chamber 16, inlet 18, and outlet 20 on a silicon wafer (not shown) by a photolithography method with a photoresist material. The etched wafer would be used to mold the chamber 16, inlet 18 and outlet 20 in a polymer such as poly(dimethylsiloxane) (“PDMS”) to form the upper layer 26. Bores 28, 30 would be made in the molded upper layer 26 to provide fluid connections to the inlet 18 and outlet 20. The lower layer 32 may be made of a biocompatible material such as glass, PDMS, polystyrene, or a metal such as titanium alloy. The lower layer 32 may be polished to maximize contact with the upper layer 26, and sterilized (e.g., by irradiation). The contact surface 34 of the upper layer 26 would be subjected to plasma treatment, and a biocompatible adhesive (e.g., a suitably prepared PDMS) would be applied. The upper and lower layers 26, 32 would then be bonded to each other via the adhesive. Although FIG. 3 illustrates a single tissue culture chamber 16, multiple chambers 16 may be provided in the upper layer to produce, for example, the series of devices 14 shown the platform 12 in FIG. 2. The tissue culture device 14 should be fabricated for transparent materials to facilitate visualization of the cells and cultured tissue.

In embodiments of the invention, the dimensions of the tissue culture chambers 16 are selected to promote the formation of 3D tissues. For this purpose, the chamber 16 may have two dimensions in the millimeter (mm) range and the third dimension in the range of hundreds of microns (μm). For example, the chamber 16 of device may have a length (L) of about 12 mm, a width (W) of about 6 mm, and a depth (D) of about 200 μm. The depth (D) of the chamber dimensions advantageously provides for the growth of cell layers having thickness of at least about 50 μm. At these dimensions, the chamber has a volume of about 10 μL. In other embodiments, the depth of the chamber is less than 1 mm and the length and width of the chamber are more than 10 times the depth of the chamber. In other embodiments, at least two of the dimensions of the chamber are less than 1 mm. Small dimensions facilitate visualization of cells and tissue, but sufficient room should be available to allow organization of multiple layers of cells.

### 3D Culturing of Cells

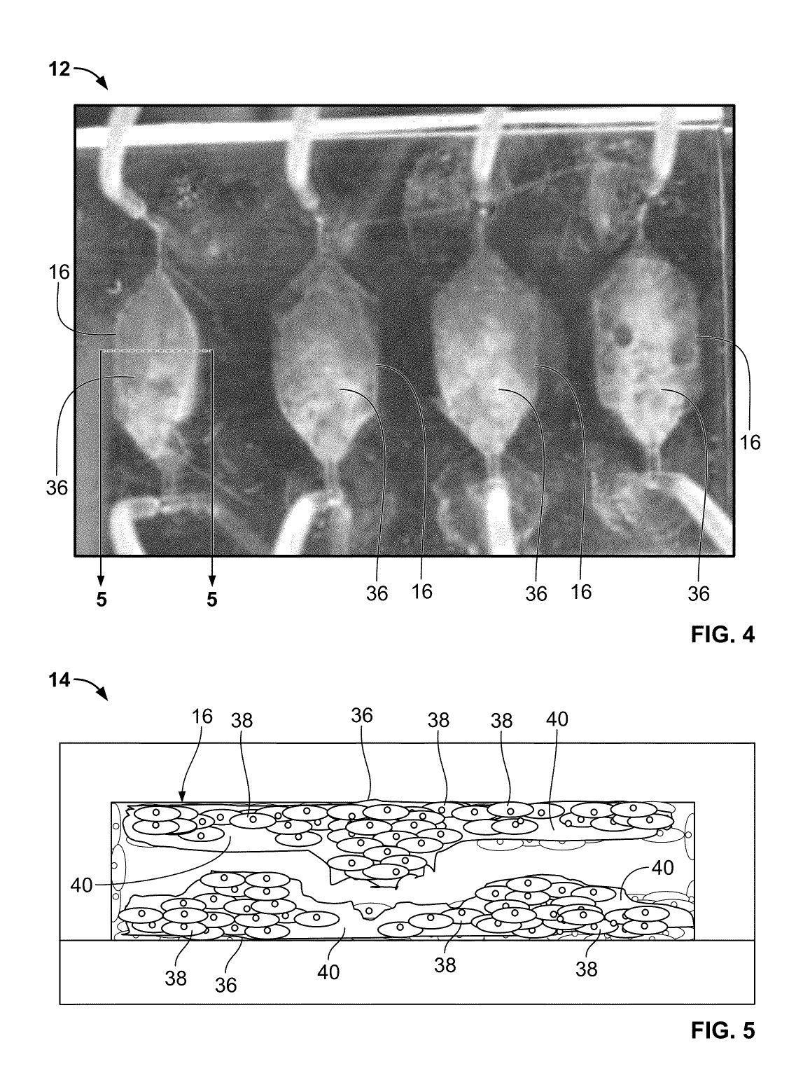

Embodiments of the present invention include methods, devices, or other means for culturing 3D tissues from immortalized cell lines and/or primary cells in microfluidic environments. FIG. 4 is a top-down view of a portion of the platform 12 of FIG. 2 with visible areas of cultured tissue 36. FIG. 5 is a schematic drawing of an exemplary cross-section of a tissue culture chamber 16 showing a cultured tissue 36 comprising cultured cells 30 in their extracellular matrix 40. The direction of flow of the culture medium or other fluid would be perpendicular to the page. The self-organization of the cultured cells 38 into a 3D tissue 36 allows a more representative simulation of perfusion of liquids in vivo than would the flow of a liquid over a two-dimensional (“2D”) monolayer cell culture. Such an arrangement yields more meaningful evaluations (e.g., evaluations of the performance of a drug on a selected tissue type) that can be used to improve the design and implementation of animal studies and human clinical trials.

The following non-limiting examples are intended to demonstrate 3D tissue culturing and drug evaluation performed according to embodiments of the present invention. The test platforms used in these examples are similar to those discussed above in relation to FIGS. 2-5. Those having ordinary skill in the art and possession of the present disclosure will recognize that the devices and methods discussed herein may be modified to investigate the responses of physiological systems other than those described in these examples, in accordance with the framework 10 of FIG. 1.

### Example 1

### Microfluidic Culturing of Breast Cancer Cells in a 3D Architecture

Human breast cancer cells (from immortalized cell line MCF-7) were incubated in a tissue culture chamber for 2 weeks with a continuous media flow at 0.8 μL/min. Initial cell attachment was seen across the entire chamber surface and continuously proliferated over time to form a 3D tissue layer having a depth of about 70-100 μm. By two weeks, nodular-like structures were seen in the culture with a small number of aggregating cells. These structures were similar to those observed in the earliest stages of tumor formation. A majority of the cells remained viable. Compared to 2D culture, MCF-7 cells grown in microfluidic chambers significantly promote cell proliferation with active morphology. By day 7, about 80% of cell confluence was seen on the microfluidic chamber surface in contrast to only about 50% on the coverslip surface of a 2D culture, suggesting that microfluidic culture not only supports the long-term culture of breast cancer cells, but also accelerates their proliferation to form 3D structures.

### Example 2

### Formation of Breast Cancer Nodules in the Presence of Adipose Stromal Cells

Breast cancer tumors exhibit dynamic and reciprocal communication between epithelial and stromal compartments during disease progression. In contrast to pure cultures of breast cancer cells, co-cultures with stromal cells stimulate breast cancer cells. Prolonged culture of MCF-7 cells with adipose stromal cells (ASCs) in the microfluidic chambers resulted in the formation of 3D breast tumor nodule-like structures. At three weeks of incubation, the nodule-like structures had become large enough to be recognized under a microscope, reaching 80-150 μm in diameter. Based on histological staining, the organization of these cells was consistent with their cancerous origin. The cells appeared to be transformed, having an increased nuclear/cytoplasmic ratio, and the cell mass was disorganized. Compared to a culture of MCF-7 alone, the inclusion of ASCs in the culture dramatically increased the nodular size (about 4-fold) and number (approximately 2-fold). The MCF-7 cells in individual nodules remained disorganized and formed 3D structures, but showed a distinct morphology different from that of MCF-7 alone, suggesting a possible phenotype change due to the microenvironment being altered by ASCs.

### Example 3

### Regulating the Progression of Breast Cancer Tissues by Controlling the Culture Medium Flow Rate

In the microfluidic tissue culture device, cells obtained nutrition from media diffused through the chamber. A flow rate that is too low cannot deliver sufficient nutrients to the cells, resulting in tissue deterioration. In an experimental protocol, MCF-7 and ASCs were cultured for 16 days at 0.8 μL/min until large nodule-like structures had formed. The medium flow rate was then lowered to 0.3 μL/min. After 3 days at the lower flow rate, a significant change was observed: in some regions, the cultured tissue began to shrink or degenerate. Hematoxylin and eosin staining of tissue cross-sections clearly showed devitalization of cells in the cultured 3D tissues, with cells being absent from various areas.

### Example 4

### Real-Time Monitoring of the Dynamic Development of Breast Cancer Tissues

Microfluidic 3D tissue culturing allows the visualization of the progression of cancer cells in the microfluidic chamber without the need for laborious histochemical analyses. The choice of transparent fabrication materials, such as glass or PDMS, as well as a chamber depth of 200 μm, allows direct visualization of cells under a microscope. With a pre-designated programmed time, images of each culture can be mapped at high resolution to show progressive development of MCF-7 cells from 2D cultured cells into 3D nodules.

### Example 5

### Utility of a Microfluidic Breast Cancer Tissue Platform for Evaluation of an Anticancer Drug

Doxorubicin (Dox), a known anticancer drug, was first tested using the microfluidic breast tissue culture described in the preceding examples. Dox was delivered to the tissue nodules at 10 nM and 1 μM, respectively, at a flow rate of 0.8 μL/min of medium via the inlet for one week. Real-time images were obtained, and a continuous progression of tumor nodules was observed in control groups without Dox treatment and the group treated with 10 nM of Dox. No obvious formation of dark nodules was observed in the groups treated with 1 μM Dox, but continuous tissue growth was noticed. The dose of 1 μM Dox is much higher than IC 50 (111±16 nM for MCF-7, producing 50% cell viability in 2D planar culture), and supposedly kills a majority of the cells in 2D culture, which would not lead to further tissue growth. In this case, the assumption was opposite to the experimental observation (i.e., the tumor tissue continuously grew), suggesting that a 3D culture has much more resistance to Dox treatment when compared to the response of a 2D culture.

### Example 6

### Utility of a Microfluidic Breast Cancer Tissue Platform for Evaluation of an Anticancer Drug

A new lytic anticancer peptide (L5) was designed and synthesized to interact with cell membranes and therefore disrupt membrane integrity and destroy cells. The peptide drug was delivered to the tissue nodules at 30 μg/mL in the medium at a flow rate of 0.8 μL/min of medium via inlet for 2 days. Upon treatment, the cells in the tissue lost their membranes. Occasionally, some nuclei were still seen in the remaining tissue matrix. However, in contrast to a 2D culture, in which all cells were killed by the peptide drug at this concentration, some MCF-7 cells survived from the treatment in the 3D microfluidic tissue culture. This may be due to a reduced rate of diffusion of the drug to the area of the surviving cells. This finding highlights the value of using the microfluidic culture model for drug assessment as it provides a more accurate representation of the 3D cellular organization encountered in vivo. It also hints at a plausible explanation to why some breast cancer therapies fail in vivo or why recurrence and multidrug resistance occurs after chemotherapy. It is noteworthy that it takes 2 weeks to create breast cancer tissues using a tissue platform according to an embodiment of the present invention in contrast to more than 3 weeks for xenografts despite laborious operation and evaluation procedures. Based on, histologic analysis, both the cultured tissues and xenografts show very similar cell morphology.

### Example 7

### Utility of a Microfluidic Breast Cancer Tissue Platform for Evaluation of Photodynamic Therapy (PDT)

A photosensitizer precursor drug, aminolevulinic acid (5-ALA) was supplemented in the culture media delivered to a 3D cultured breast cancer tissue (MCF-7), and allowed to infiltrate the tissue via perfusion for 4 hours to emulate microinjection. After cellular uptake, 5-ALA was converted into photosensitive protoporphyrin (PpIX). Upon illumination with broadband halogen light for 1 minute, PpIX absorbed light energy, became excited and induced elevated formation of reactive oxygen species (ROS) by transferring the energy to neighboring O₂ molecules. High ROS concentrations led to cell destruction. To determine the extent of cell damage, the culture was stained with a live/dead staining kit (Sigma) after overnight culture and examined with a Leica confocal microscope. After PDT treatment, about 50% of the cells in the 3D tissues were killed, whereas about 70% were killed in a 2D culture. This observation indicates that the tissue culture platform is suitable for PDT evaluation and suggests that monolayer cultures exaggerate drug potency, as perfusion is not emulated in 2D cultures.

### Example 8

### Microfluidic Osteoblast Tissue Culture

Mouse calvarial preosteoblast cells (MC3T3-E1) were seeded into multiple microfluidic tissue culture chambers and provided with a continuous flow of culture medium. After 5 weeks of culture, the thickest part of the tissue in the culture chamber was measured to be about 150 μm. Most of the tissue structures formed close to the bottom glass surface. Formation of the 3D tissue began with the adhesion and spreading of osteoblasts on the bottom surface of the culture chambers and formed a confluent layer by Day 4. The cells started to migrate to side walls and the edges of the top surfaces around Day 5 and to the center of the top surface by Day 7. After the cells proliferated to occupy all of the available surfaces they started to form multiple cell layers around Day 10.

The cells present in the upper layers of the multilayer structures were observed to be round in shape. Also, after about Day 10, significant local cell aggregations were observed. An exemplary 3D cell aggregate was formed by the shrinkage of the upper layer cells while the cells adhered on the chamber surface remained on the surface. At the aggregate boundary, it appeared that the cells remaining on the surface were significantly stretched. This observation suggested that 3D cell aggregates were formed by local contractions of the multilayer cells. After the 3D aggregates were formed, the cells remaining on the surface continued to proliferate and form new 3D aggregates. This resulted in the accumulated growth and densification of the tissue structures into the interior space of the microfluidic chambers. By Day 16, the 3D tissue formation was evident in all areas of the chambers.

SEM images of the 5-week tissue sample showed the presence of randomly oriented collagen fibers and calcium-rich minerals in the extracellular matrix. The diameter of the collagen fibers was typically a few tens of nanometers, although some fibers approached 100 nm. The minerals appeared as curly flower-like clusters of 1-2 μm crystals. The Ca/P weight ratio was estimated to be about 1.5 by analyzing the relative intensity of the calcium to phosphorous peaks in an EDS spectrum. Calcium deposition as measured by alizarin red staining increased with increasing culture time.

The observed 3D tissue structures were composed of osteoblasts that were round in shape and embedded within the extracellular matrix of collagen fibers and calcium phosphate crystals with random orientation and distribution. This morphology is similar to what is commonly referred to as “primary bone tissues,” which are produced in a fetus or during the earliest healing stage after bone fracture. Primary bone tissues are made of randomly oriented coarse collagen fibers, calcified materials and osteocytes. Remodeling of the primary tissue over a few years is required to form secondary bone tissue with hierarchically complex multi-scale structures.

It was also observed that: (i) significant cell proliferation beyond confluent layers led to multilayer formation and local 3D cell aggregation; (ii) the morphological shape of cells changed from spread to round during this transition; and (iii) the accumulation of local cell aggregations resulted in the development of dense tissue-like structures that eventually filled the chambers. It appears that the cell aggregations were accompanied by significant contractions, which were sufficient to cause the detachment and movement of the micropatterns.

### 3D Microfluidic Tissue Culture Devices

Various embodiments of the 3D microfluidic tissue culture devices 12 may be employed to grow, maintain and assess cells in each physiological system. The microfluidic tissue culture devices discussed with respect to FIGS. 2-5 and the foregoing examples represent relatively simple embodiments of the devices and platform. In other embodiments of the present invention, the devices are more complex in their construction, allowing a more sophisticated evaluation of tissue responses to drugs and other treatments. Such devices may be interconnected according to the framework disclosed in FIG. 1 to simulate entire physiological systems.

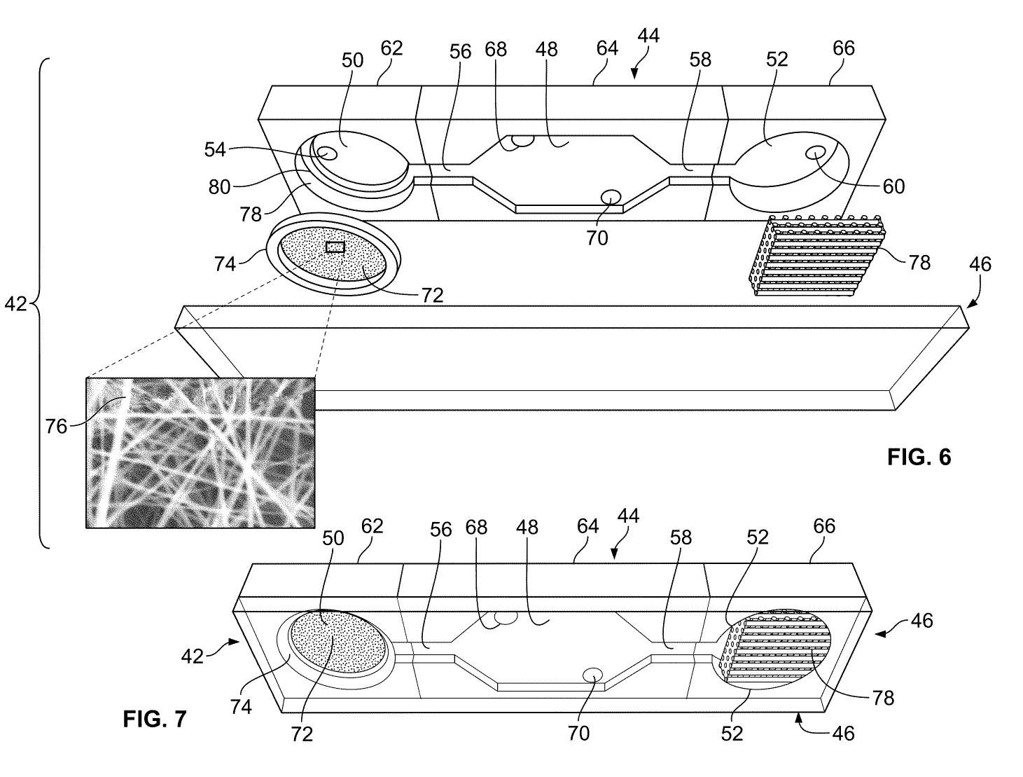

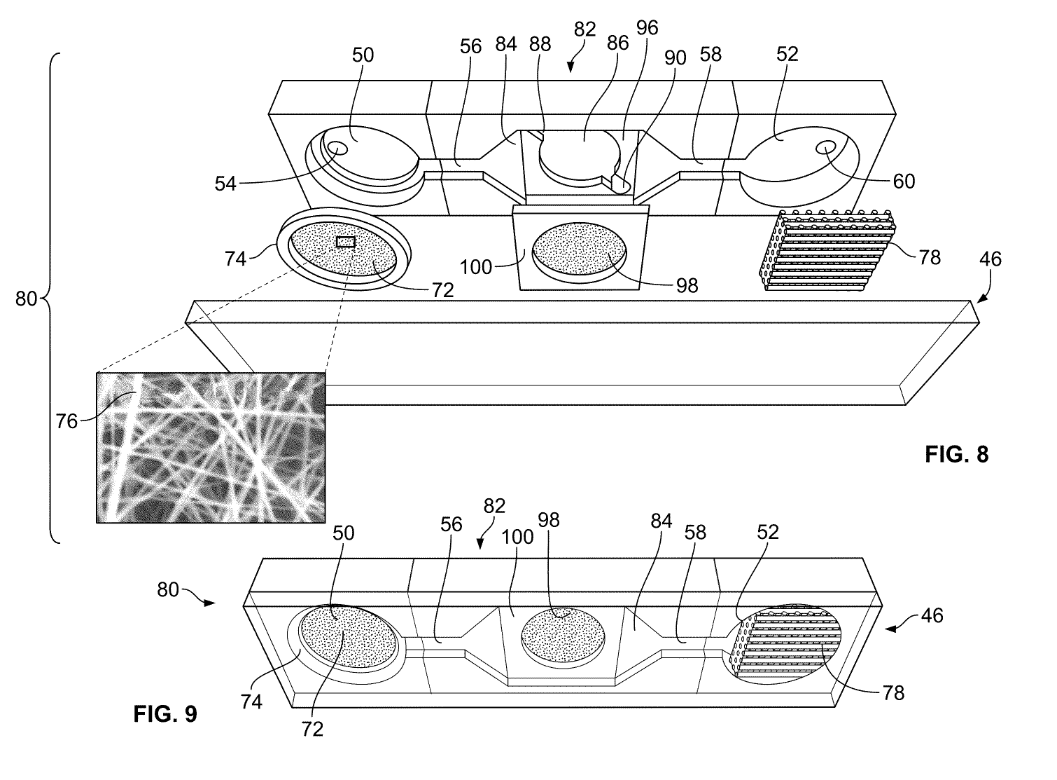

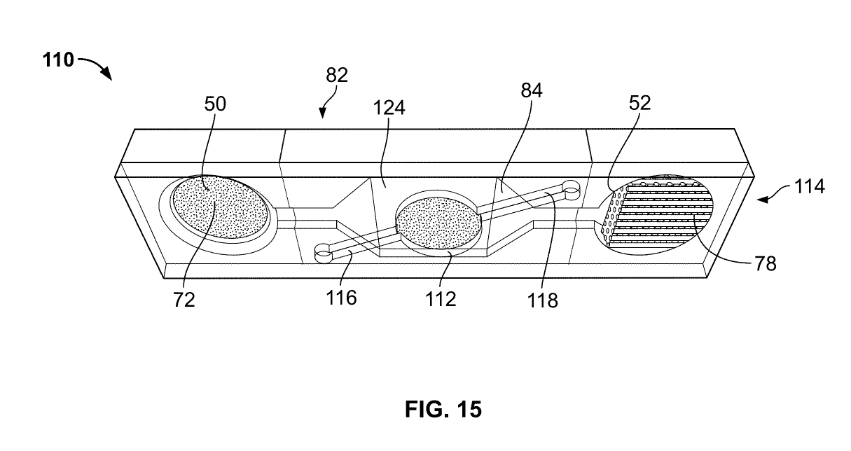

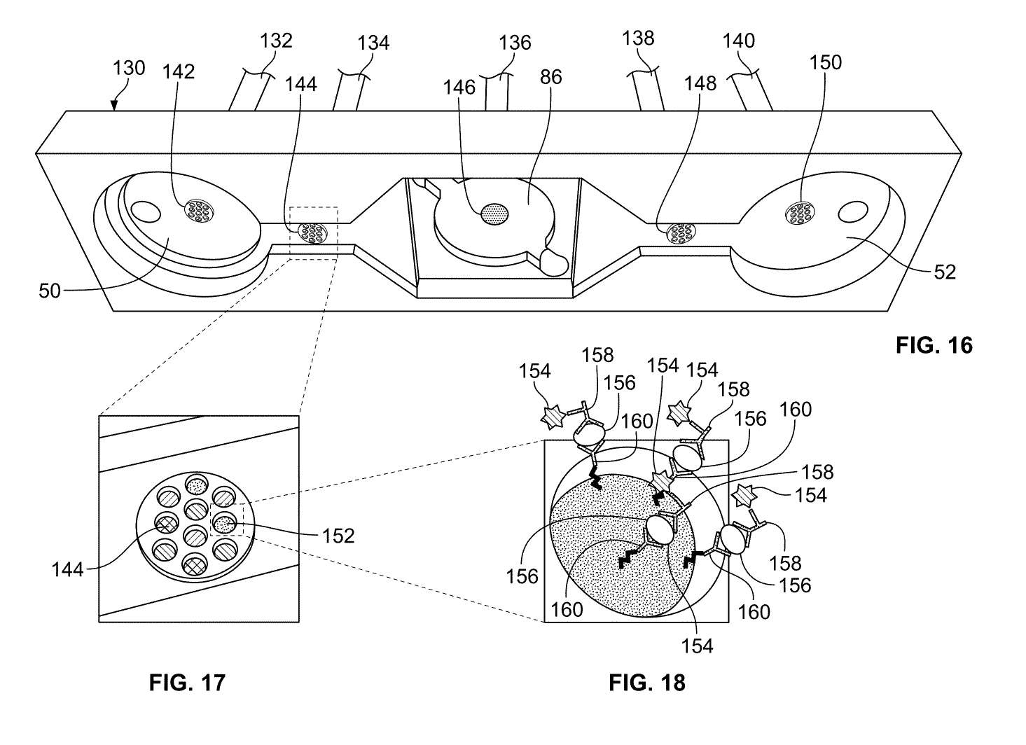

The present embodiment encompasses numerous embodiments of tissue culture devices and platforms that build on the basic tissue culture devices disclosed herein. Representative embodiments of such basic tissue culture devices which are discussed hereinbelow are identified as Type 1 devices (FIGS. 6 and 7), Type 2 devices (FIGS. 8 and 9), and Type 3 devices (FIGS. 15 and 16). It is understood that other embodiments of the present invention include devices that build on the Type 1, Type 2, and Type 3 devices, but may vary in the size, shape and configurations of chambers, and the interconnectivity between the chambers. The description of the Type 1, Type 2 and Type 3 devices hereinbelow is not intended to limit the scope or size, shape or configurations of chambers, or the interconnectivity between chambers of the devices.

FIGS. 6 and 7 are schematic drawings of a Type 1 device 42 in a partially disassembled state (FIG. 6) and a fully assembled state (FIG. 7). The upper layer 44 of the device 42 may be made of PDMS and the lower layer 46 may be made of glass or other materials, as discussed with respect to the device of FIG. 3. There are three different chambers in each Type 1 device: i) a central 3D microfluidic tissue culture chamber 48 at the middle position in the device 42, ii) a pre-chamber 50 (intended in some embodiments to simulate a microvascular system), which is positioned on the left side of the device 42 as viewed in FIG. 6, and iii) a post-chamber 52 (intended in some embodiments to simulate a lymphatic node) which is positioned on the right side of the tissue culture chamber 48 as viewed in FIG. 6. In some embodiments of a Type 1 device 42, fluid enters the pre-chamber 52 through a bore 54, flows from left to right (i.e., upstream to downstream) through inlets 56 and outlets 58, and exits the device 42 through the bore 60 in the post-chamber 52. The upper layer 44 may be formed as a single piece containing the tissue culture chamber 48, the pre-chamber 50, and the post-chamber 52, or each chamber 48, 50, 52 may be formed in an individual piece of the upper layer (e.g., pieces 62, 64, 66), which are then joined to each other using adhesive or some other method.

The 3D microfluidic tissue culture chamber 48 may have an elongated hexagonal shape with dimensions as discussed above with respect to the device of FIG. 3. In practice, the tissue culture chamber 48 is the location in the device where 3D tissues of selected cell types are cultured to represent a component of a physiological system. In an embodiment, the tissue culture chamber 48 may be designed for accommodating 3D-structured tissues such as musculoskeletal (bone, cartilage, muscle), circulatory (cardiac) and reproductive (embryo) tissues. In embodiments of the Type 1 device 42, the tissue culture chamber 48 may also have bores 68, 70 may serve as inlets for cell seeding and as inlets or outlets for streaming culture medium.

Within the Type 1 device 42, the central 3D microfluidic tissue culture chamber 48 is connected on its left (upstream) side to the pre-chamber 50, which, in some embodiments, represents the microvascular system from which the flow of fluid originates in the device 42. In the illustrated embodiment of the Type 1 device 42, the pre-chamber has a circular shape be divided into upper and lower chambers by a mesh 72 (not visible in FIG. 6 or 7). The circular pre-chamber may be 5 mm in diameter and with the upper and lower chambers, having respective depths of 0.2 mm and 0.4 mm.

The mesh 72 is a semi-permeable biomimetic nanofiber mesh 72 which is collected on a PDMS frame 74, or a frame made of another biocompatible material. The nanofibers 76 may be collected by placing the frame 74 on a grounded foil and electroforming the fibers 76 over the frame 74. The structure of the mesh 72 can thus be controlled during assembly to provide a mesh 72 having the desired permeability. The nanofibers 76 may be made of a biocompatible material to which cells will adhere. Such materials include biocompatible synthetic polymers (e.g., polycaprolactone) with or without extracellular matrix (ECM) proteins (e.g., type I collagen or laminin). Tissue growth factors (e.g., vascular endothelial growth factors) may also be included with the synthetic polymers or ECM proteins.

The frame 74 is arranged to fit closely within the pre-chamber 50 at the inner surface 78 of the pre-chamber. A ledge 80 may be provided in the pre-chamber 50 to position the frame 74 such that the nanofiber mesh 72 maintains the desired depths of the lower and upper chambers (not visible in FIG. 6 or 7).

In embodiments of the Type 1 device, endothelial cells are cultured on the nanofiber mesh 72 using standard culturing methods, or modifications thereof, to reconstruct and simulate the semi-permeable nature of vascular capillary membrane. These endothelial cells may be cultured on the membrane 72 before the pieces of the upper layer 44 are assembled, although they may be cultured in embodiments where the upper layer 44 is a single piece. In embodiments of the present invention, culturing of the epithelial cells should be completed before 3D tissue culturing begins. In an exemplary culturing method, the cells are seeded onto the nanofiber mesh 72 after the Type 1 device 42 is assembled. Epithelial cells suspended in culture media are added into the pre-chamber 50 via bore 54 and then allowed to settle and attach to the surface of the mesh 72 for 1-2 hours. Cells have a high tendency to attach to the fibers 76 due to the morphological and compositional similarities between the fibers 76 and the native ECM fibrils. After cell seeding, culture media is allowed to flow through the pre-chamber 50, and the attached cells remain on the mesh 72.

The circular post-chamber 52 is placed at the exit stream to the right (downstream) of the tissue culture chamber 48, which, in some embodiments of the Type 1 device 42, represents the lymphatic system. The post-chamber 52 may have a circular shape with a diameter of 5 mm and a depth of about 0.6 mm. The post-chamber 52 may be filled with a network of reticular microfibers 78 to mimic the reticular connective tissue in lymph nodes. Phagocytes (macrophages) and lymphocytes (T cells) may be seeded and anchored on the reticular network 78 using methods such as those discussed with respect to the seeding of epithelial cells on the mesh 72 in the pre-chamber 50. In embodiments of the Type 1 device 42, the bore 60 may serve as an inlet for cell seeding.

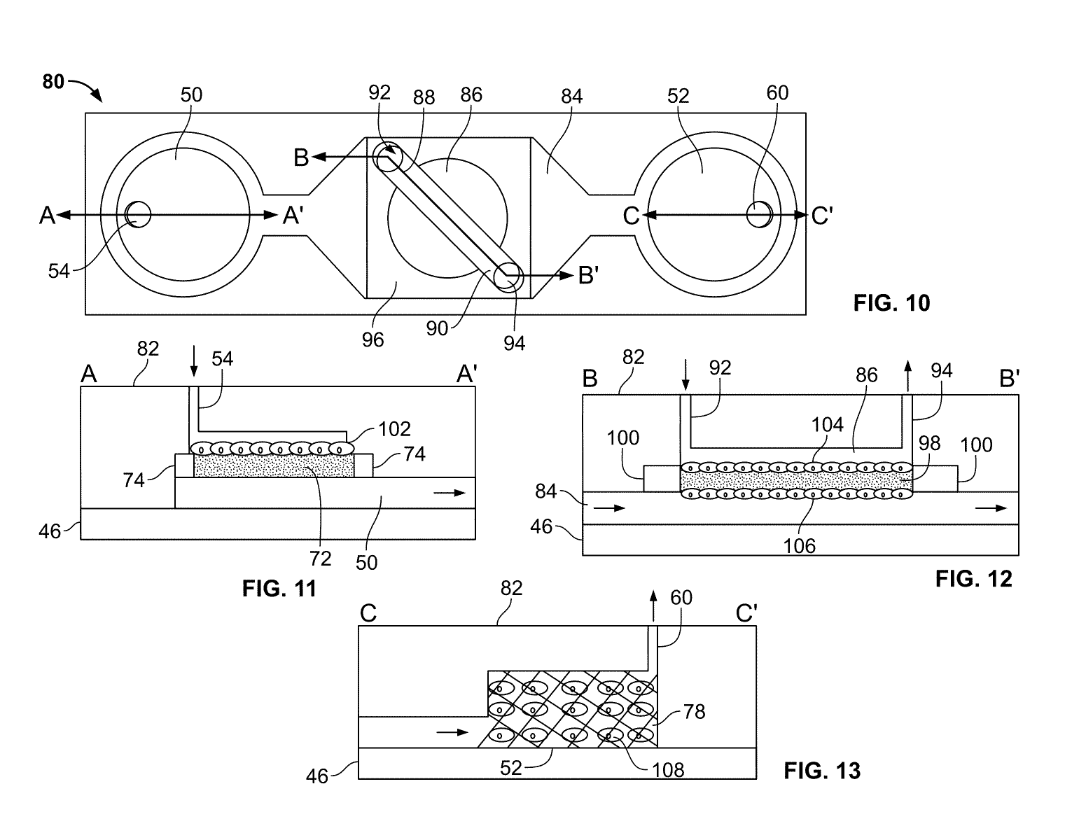

FIGS. 8 and 9 are schematic drawings of an embodiment of a Type 2 device 80 in a partially-disassembled state (FIG. 8) and an assembled state (FIG. 9). A bottom view of the upper layer 82 of the embodiment of the Type 2 device 80 is presented in FIG. 10. Elements of the embodiment of FIGS. 8, 9 and 10 discussed herein that are common to the Type 2 device 80 and the embodiment of the Type 1 device 42 of FIGS. 6 and 7 and serve the same functions as those elements of Type 1 device 42 are labeled with the same reference numbers used in FIGS. 6 and 7. Descriptions of such common elements may be found in the discussion of the Type 1 device 42.

Referring to FIGS. 8, 9, and 10, embodiments of the Type 2 device 80 may be distinguished from embodiments of the Type 1 device 42 by the arrangement of the central 3D microfluidic tissue culture chamber 84. While the tissue chamber 84 of the Type 2 device 80 is similar to that of the tissue culture chamber 16 of the Type 1 device 42, the upper layer 82 of the Type 2 device 80 has an additional chamber 86 adjacent to the tissue culture chamber 84. The additional chamber 86 may be provided with an inlet 88 and an outlet 90 for cell seeding and fluid flow, as well as bores 92, 94 (visible in FIG. 10), to provide fluid communication between the additional chamber 86 and the remainder of the platform. In an embodiment, the additional chamber 86 is circular and has a diameter of 3 mm and a depth of 0.2 mm.

The additional chamber 86 (hereinafter, “the recessed chamber 86”) is recessed into the upper layer 82 within a receptacle 96 in the tissue culture chamber 84. A semi-permeable biomimetic nanofiber mesh 98 which is collected on a PDMS frame 100, or a frame made of another biocompatible material, is provided to separate the recessed chamber 86 from the tissue culture chamber 84. The frame 100 and receptacle 96 are arranged such that the frame 100 fits closely into the receptacle 96 without substantially reducing the volume of the tissue culture chamber 84.

The semi-permeable biomimetic nanofiber mesh 98 may be similar to the nanofiber mesh 72 of the aforementioned Type 1 device 42, and mimics the basement membrane structure of epithelial and endothelial cell layers, providing engineered tissues as a physiologically-relevant model to assess drug response. In an embodiment, the inlet 88 and outlet 90 of the recessed chamber 86 can be used to seed tissue cells onto the mesh 98 as well as apply a secondary stream of medium with supplements for any particular tissue type grown on the mesh 98.

FIGS. 11, 12, and 13 are schematic cross-sections of the Type 2 device 80 of FIGS. 8, 9, and 10, showing cross-sections of the pre-chamber 50 (FIG. 11), tissue culture chamber 84 and recessed chamber 86 (FIG. 12), and post-chamber 52 (FIG. 13). Tissues are represented schematic drawings of cells 102, 104, 106, 108 in the FIGS. 11, 12, and 13. Referring to FIG. 11, in an embodiment of the present invention, epithelial cells 102 are seeded onto the mesh 72 in the pre-chamber 50 and cultured as discussed with respect to the aforementioned Type 1 device 42 to mimic a microvascular system. Referring to FIG. 12, in an embodiment of the present invention, the tissue culture chamber 84 and recessed chamber 86 of a Type 2 device 80 can be used to grow tissues with barrier functions (e.g. gastrointestinal (GI track, liver, intestine, stomach), and urinary (kidney) tissues), by seeding the membrane 98 with a first type of cell 104 in the recessed chamber and culturing the first type of cell 104, and by culturing a second type of cell 106 on the membrane 98 in the tissue culture chamber 84. For example, the first type of cell 104 may be endothelial cells and the second type of cell 106, which are to be cultured to form a 3D tissue, may be hepatocytes. In such an example, the portion of the device 80 shown in FIG. 12 may be used to mimic liver function. Still referring to FIG. 12, in another embodiment of the present invention, the Type 2 device 80 may be utilized for building tissues representing integumentary (skin) and respiratory (lung) systems, For that purpose, the recessed chamber 86 may be provided with a flow of air from the bore 92 to the bore 94 to emulate the air/liquid interface present in the integumentary and lung tissues. In an embodiment of the present invention, the microfiber mesh 78 in the post-chamber 52 is seeded with immune cells 108 as discussed with respect to the aforementioned Type 1 device 42 to mimic a lymphatic system.

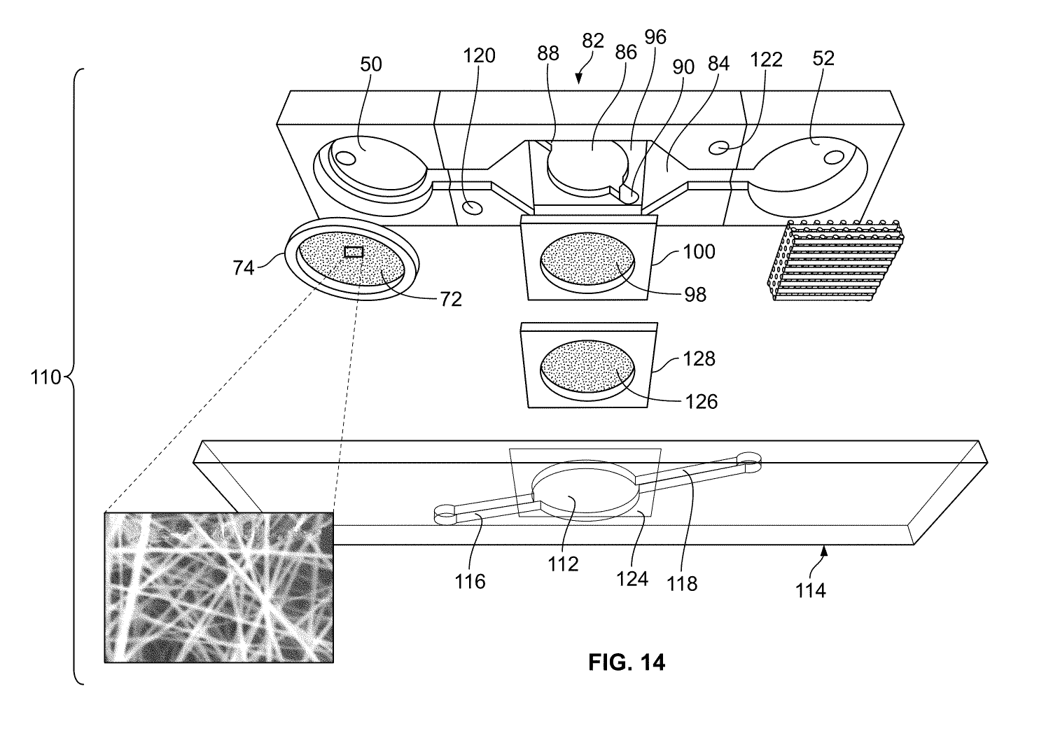

FIGS. 14 and 15 are schematic drawings of an embodiment of a Type 3 device 110 in a partially-disassembled state (FIG. 14) and an assembled state (FIG. 15). Elements of the embodiment of FIGS. 14 and 15 that are common to the Type 3 device and the embodiments of the Type 1 device 42 of FIGS. 6 and 7 or the Type 2 device 80 of FIGS. 8, 9, and 10, and serve the same functions as those elements, are labeled with the same reference numbers used in FIGS. 6-10. Descriptions of such common elements may be found in the discussions of the Type 1 device 42 and Type 2 device 80.

Referring to FIGS. 14 and 15, embodiments of the Type 3 device 110 may be distinguished from embodiments of the Type 2 device 80 by the presence of a second recessed chamber 112 that is recessed into the lower layer 114 of the Type 3 so as to be aligned with the tissue culture chamber 84. The recessed chamber 112 may be provided with an inlet 116 and an outlet 118 for cell seeding and fluid flow, as well as bores 120, 122 through the upper layer 82 to provide fluid communication between the recessed chamber and the remainder of the platform. In an embodiment, the recessed chamber 112 may be circular with a diameter of 3 mm and a depth of 0.2 mm. The recessed chamber 112 may be formed by an etching process, if, for example, the lower layer 114 is made from glass, or a molding process, if, for example, the lower layer 114 is made of PDMS.

The recessed chamber 112 is recessed into the lower layer 114 within a receptacle 124 generally aligned with the receptacle 96 in the upper layer 82 of the Type 3 device 110. A semi-permeable biomimetic nanofiber mesh 126 which is collected on a PDMS frame 128, or a frame made of another biocompatible material, is provided to separate the recessed chamber 112 from the tissue culture chamber 84. The frame 128 and receptacle 124 are arranged such that the frame 128 fits closely into the receptacle 124 without substantially reducing the volume of the tissue culture chamber 84.

The semi-permeable biomimetic nanofiber mesh 126 may be similar to the nanofiber meshes 72, 98 of the aforementioned Type 1 and Type 2 devices 42, 80, and mimics the basement membrane structure of epithelial and endothelial cell layers, providing engineered tissues as a physiologically-relevant model to assess drug response. In an embodiment, the bores 120, 122 can be used to seed tissue cells onto the mesh 126 as well as apply a secondary stream of medium with supplements for any particular tissue type grown on the mesh 126.

Embodiments of the Type 3 device 110 can be applied to grow tissues with barrier functions, e.g. gastrointestinal (GI track, liver, intestine, stomach), and urinary (kidney) tissues. Embodiments of the Type 3 device can also be utilized for building tissues representing integumentary (skin) and respiratory (lung) systems. For that purpose, one or both of the recessed chambers 86, 112 may be provided with a flow of air from the respective inlet 88, 116 to the respective outlet 90, 118 to emulate the air/liquid interface present in those tissues. Because two opposing membranes 98, 126 are present, two opposing barrier functions may be mimicked, rather than just one.

### On Board Detection Systems for the Assessment of Drugs, Biomarkers and Cells

In certain embodiments of the present invention, the platform (not shown) may be provided with an on-board detection system for sensing and/or visualization of drugs, biomarkers, cells, and tissues. The on-board detection system enables on-board microarray multiplexed analysis of drugs, biomarkers of toxicity and efficacy, and cellular imaging.

FIG. 16 is a schematic illustration of an upper layer 130 of an embodiment of a Type 2 device for use in a platform having such an onboard detection system. Elements of the embodiment of FIG. 16 that are common to the embodiment of the upper layer 130 of the Type 2 device of FIG. 16, and the embodiments of the Type 1 device 42 of FIGS. 6 and 7 or the Type 2 device 80 of FIGS. 8, 9, and 10, and serve the same functions as those elements, are labeled with the same reference numbers used in FIGS. 6-10. Descriptions of such common elements may be found in the foregoing discussions of the Type 1 device 42 and Type 2 device 80.

Referring to FIG. 16, the on-board detection system (not shown) includes a multiplicity of optical fiber bundles 132, 134, 136, 138, 140, whose distal ends 142, 144, 146, 148, 150 are positioned at strategic positions within the upper layer 130 selected for detecting various aspects of the simulated physiological systems. In the illustrated embodiment, the distal ends 142, 150 of optical fiber bundles 132, 140 are positioned in the pre-chamber 50 and post-chamber 52, respectively, and the distal ends 144, 148 of optical fiber bundles 134, 138 are positioned in the inlet 56 and outlet 58 of the tissue culture chamber 84. The distal end 146 of optical fiber bundle 136 is positioned in the recessed chamber 86.

Each optical fiber bundle 132, 134, 136, 138, 140 may comprise tens of thousands of individual fibers. For clarity, only 10 fibers (represented as shaded circles in FIG. 17) are shown in the distal end 144 of optical fiber bundle 134 that is depicted in the enlarged view illustrated in FIG. 17. Each fiber may be individually functionalized for a specific purpose. For example, fiber 152 of FIG. 17, depicted in an enlarged view in FIG. 18, may be functionalized to detect biomarkers 154 secreted by the cultured cells. Multiplex detection of the secreted biomarkers 154 is enabled by encoded biomarker-capture microspheres 156 (i.e., coated with specific antibodies 158 on the surface of the microspheres 156) and Au nanoparticle-decorated microspheres for SERS analysis (not shown). The microspheres 156 are tethered to the optical fiber by linking molecules 160, and may be distributed so that a single microsphere 156 resides at the tip of each fiber. These antibody-coated microspheres 156 selectively and specifically bind the targets 154 (biomarkers). Detection occurs by flowing fluorescently labeled secondary antibodies past the distal end of the fiber optic bundle 134. Alternatively, a competitive inhibition assay can be established where antibody-coated microspheres bound to synthetically made and fluorescently tagged biomarkers are distributed and reside at the tip of each fiber and are displaced when the natural biomarker produced by the cell culture is available to for specific competition resulting in a decrease in florescent signal.

The optical fiber bundle 136 associated with the recessed chamber 86 has a specific configuration that enables real-time confocal imaging of live cells and tissues as they respond to drug treatments or other changes in their culture medium. Confocal imaging is realized through incorporation of a variable focal length microlens (not shown) at the distal end of the optical fibers, between the growing tissue and the distal end 146 of the fiber bundle 136. In such an arrangement, each of the individual fiber elements in the bundle corresponds to a pixel in the resultant optical image. Fluorescent imaging of the tissue may be used.

An on-board detection system according to the embodiment of the present invention discussed above has a number of practical and beneficial attributes, such as: alternatives to the common off-board ELISA method, which requires large sample volumes and long analysis times for a single analyte; on-board multiplexing assays for assessment of secreted biomarkers; on-board SERS and label-free measurements of drugs; on-board real time endoscopic imaging of tissue morphology and migrating cells; small sample volume analysis; small footprint for on-board integration in portable systems; time/space-resolved In-Vivo measurements within key compartments of each device; ease of use, image analysis and data acquisition; and comparatively low cost

It should be appreciated that the present invention provides numerous advantages over current platforms. For instance, a platform comprising the devices of the present invention provides a low-cost, scalable facility that simulates major human physiological system functions for rapid evaluation of drug safety, efficacy and pharmacokinetics prior to clinical trials. The platform can be used for assessment of individual drugs or in personalized medicine applications where all cells are derived from a single patient. Since the platform uses physiologically-relevant integrated cells and tissues, it can be used to improve or replace the current practice of animal testing of drugs prior to human clinical drug trials, which typically fails to predict drug response in humans. The platform integrates the circulatory and lymphatic systems in a physiologically correct manner, and has flow paths and connections that mimic the pathways and interactions of the major organ.

It will be understood that the embodiments described herein are merely exemplary and that a person skilled in the art may make many variations and modifications without departing from the spirit and scope of the invention. For example, microelectrode arrays made of conductive materials, such as platinum or gold, can be provided on surfaces of the tissue culture chambers of the Type 1 and 2 devices to stimulate and record electrophysiological activity of neurons in neural tissues. Such electrodes may be formed by inkjet printing commercially available inks onto the selected surface. All variations and modifications that may be recognized by those having ordinary skill in the art are intended to be included within the scope of the invention, as defined by the claims presented below.

We claim:

1. A microfluidic system for evaluating the effects of a biologically active compound on a tissue, comprising:
at least first and second devices for culturing three-dimensional tissues from cells, each of said devices including a respective tissue culture chamber, each of said tissue culture chambers having an inlet and an outlet for facilitating fluid flow through said each of said tissue culture chambers;
a conduit fluidly connecting said outlet of said tissue culture chamber of said first device to said inlet of said tissue culture chamber of said second device; and
a microfluidic pump arranged for providing a fluid at a microfluidic flow rate to said inlet of said tissue culture chamber of said first device.

2. The system of claim 1, further comprising a three-dimensional cultured tissue within one of said tissue culture chambers, wherein said cultured tissues are cultured within said one of said tissue culture chambers from cells seeded into said one of said tissue culture chambers.

3. The system of claim 2, wherein at least some of said cells are diseased or otherwise abnormal cells collected from a human patient

4. The system of claim 2, wherein said cells are derived from an animal organ representative of a physiological system and said cultured tissue is representative of the physiological system, and wherein said microfluidic pump, said conduit, and said respective tissue culture chambers are arranged such that the microfluidic flow rate and the residence time of the fluid in at least one of said tissue culture chambers represent the cardiac output and residence time present in the physiological system under normal homeostatic physiological conditions

5. The system of claim 1, wherein at least one of said tissue culture chambers has dimensions of length, width, depth, and at least one of said dimensions is less than about 1 millimeter.

6. A microfluidic device for culturing a three-dimensional tissue and evaluating the effects of a biologically active compound on such a tissue, comprising:
a first chamber for culturing tissues in three-dimensions, said first chamber having a first inlet and a first outlet for facilitating fluid flow through said first chamber, and having dimensions of length, width, depth, at least one of said dimensions being less than about 1 millimeter;
a second chamber having a second inlet and a second outlet for facilitating fluid flow through said second chamber, said second chamber also having a first semipermeable membrane therein which divides said second chamber into a first subchamber fluidly connected with said second inlet and a second subchamber fluidly connected with said second outlet, said first semipermeable membrane arranged such that cells seeded into said first subchamber adhere to said first semipermeable membrane and proliferate thereupon, said first semipermeable membrane being permeable to water; and
a third chamber having a third inlet and a third outlet for facilitating fluid flow through said third chamber, said third chamber also having microfibers therein, said microfibers arranged such that cells seeded into said third chamber adhere to said microfibers and proliferate thereamong, wherein said second outlet of said second chamber is fluidly connected with said first inlet of said first chamber and said first outlet of said first chamber is fluidly connected with said third inlet of said third chamber.

7. The microfluidic device of claim 6, further comprising a fourth chamber having a fourth inlet and a fourth outlet for facilitating fluid flow through said fourth chamber, a second semipermeable membrane separating said fourth chamber from said first chamber, said second semipermeable membrane having first and second surfaces opposite each other, said second semipermeable membrane being arranged such that cells seeded into said fourth chamber attach to and proliferate on said first surface and cells residing in said first chamber attach to and proliferate on said second surface, said second semipermeable membrane being permeable to water.

8. The microfluidic device of claim 7, further comprising a fifth chamber having a fifth inlet and a fifth outlet for facilitating fluid flow through said fifth chamber, a third semipermeable membrane separating said fifth chamber from said first chamber, said third semipermeable membrane having third and fourth surfaces opposite each other, said third semipermeable membrane being arranged such that cells seeded into said fifth chamber attach to and proliferate on said third surface and cells residing in said first chamber attach to and proliferate on said fourth surface, said third semipermeable membrane being permeable to water.

9. The device of claim 6, further comprising a three-dimensional cultured tissue within said first chamber, wherein said cultured tissue is cultured within said first chamber from cells seeded into said first chamber.

10. The device of claim 9, further comprising cells proliferating on said first semipermeable membrane of said second chamber, whereby said second chamber is physiologically representative of a microvascular system.

11. The device of claim 9, further comprising cells proliferating among said microfibers of said third chamber, whereby said third chamber is physiologically representative of a lymphatic system.

12. The device of claim 6, further comprising at least one optical fiber having an end located in a location selected from the group consisting of one of said chambers, an inlet of one of said chambers or an outlet of one of said chambers, said end of said at least one optical fiber selected from the group consisting of an end that has a microlens for visualization of cells and cultured tissue, an end that is functionalized to detect biomarkers secreted by cells, and an end that is functionalized to detect the biologically active compound.

13. A method of evaluating the effects of at least one biologically active compound on a physiological system, comprising the steps of:
seeding cells derived from an animal organ representative of the physiological system into a tissue culture chamber;
passing a culture medium through said tissue culture chamber at a microfluidic rate such that the cells proliferate and self-organize into a three-dimensional tissue;
changing the composition of the culture medium, including the step of adding an amount of the at least one biologically active compound into the culture medium; and
observing changes in the cells and cultured tissue over time.

14. A method of preparing a personalized treatment regimen for a patient having diseased or abnormal tissues, comprising the steps of:
(a) seeding a first group of cells derived from a diseased or otherwise abnormal tissue from a human patient into a first tissue culture chamber;
(b) seeding a second group of cells into a second tissue culture chamber, said second group of cells derived from one of a healthy tissue from the human patient and a reference human cell population representative of the healthy tissue;
(c) passing a culture medium through said first and second tissue culture chambers at a microfluidic rate such that the first and second groups of cells proliferate and self-organize respectively into first and second three-dimensional tissues;
(d) changing the composition of the culture medium over time, including the step of adding a quantity of at least one biologically active compound into the culture medium;
(e) observing changes in the first and second groups of cells and the first and second cultured tissues over time and recording the observed changes that are relevant to developing a treatment regimen for the human patient for relief of the diseased or otherwise abnormal tissue; and
(f) selecting the treatment regimen for the patient after analyzing the recorded observations.

15. The method of claim 14, including the further step of repeating all of the steps (a) through (e) until sufficient observations are recorded to support selection of the treatment regimen, while changing one or both of the composition of the at least one biologically active compound and the quantity of the at least one biologically active compound during at least some of the repetitions of step (d).
